(19) United States
(12) Patent Application Publication
(10) Pub. No.: US 2009/0104717 A1
(43) Pub. Date: Apr. 23, 2009

(54) System to Reduce Incubation Time in Immunological Testing Using Enhanced Microwaves

(76) Inventors: Wayne G. Love, Glen Ellyn, IL (US); Janos Luka, New Windsor, MD (US)

Correspondence Address: GREENBERG & LIEBERMAN, LLC, 2141 WISCONSIN AVE, N.W., SUITE C-2, WASHINGTON, DC, 20007, US

(21) Appl. No.: 12/257,197

(22) Filed: Oct. 23, 2008

Related U.S. Application Data
(60) Provisional application No. 60/999,948, filed on Oct. 23, 2007.

(51) Int. Cl. G01N 33/536

(52) U.S. Cl. 436/536

(57) ABSTRACT

A system for shortening incubation times on immunoassays employs energy that is applied to the assays. Energy is applied in careful gradations so that chemical bonds are not broken. If polyclonal antibodies, as opposed to monoclonal antibodies, are worked with, then because the bonds are stronger, cooling is employed in combination with the extra energy delivered.

### PRIORITY STATEMENT

This is a nonprovisional patent application of provisional patent application No. 60/999,948 filed on Oct. 23, 2007, and priority is claimed thereto.

### FIELD OF THE INVENTION

The present invention is a method to reduce incubation time in immunological testing by using enhanced microwaves to shorten the incubation times with resolution equal to or better than traditional incubation methods and times.

### BACKGROUND OF THE INVENTION

The present invention has a number of benefits relating to reducing the incubation time in immunological testing, particularly with the use of enhanced microwaves. Immunological testing as termed here means testing of blood, blood serum, bodily fluids or tissues for antibodies, antigen, enzyme reactions, In Situ Hybridization (also called FISH) or similar Immunological reactions common to Enzyme-linked Immunosorbent Assays (ELISA) (ELISA assays are also sometimes called Microtieter assays), protein blotting methods (commonly Western Blot, Immunohistochemistry or other Immunopercipitation methods. It does not include histostaining to improve imaging of cells performed on histological embedded tissues. Microwaves have been used for accelerating these types of processes for some time. Immunological testing includes the testing for HIV, certain cancers, allergies, diabetes and other diseases and is one of the most common laboratory testing performed in the medical field.

In the above Immunological testing, antigen or antibodies, proteins or enzymes are mixed and react (frequently bonding in antibody-antigen pairs) and must be given a period of time to react, called the “incubation time”. While there may be multiple incubation periods in each immunological assay, most of the incubation periods are 1 hour or longer. For example, there maybe 2 or 3 incubation periods within a given ELISA assay each at least 1 hour long. Typical incubation is done at room temperature or at slightly elevated temperatures in incubation ovens generally at 37° C. However the antibodies and/or antigens become denatured in temperatures above around 45° C.

Resolution is important in Immunological testing. Higher resolution means lower levels of undesirable antigens can be detected and equate to earlier detection of disease or viruses that can be treated with higher success rates than those detected later. Essentially, the assays combine certain antibodies and antigen and then the results are determined through the number of these combinations as detected in different methods depending on the technique used.

Microwaves

Microwaves have long been used for foods and in industrial, laboratory and medical applications because they offer a unique method of processing material quickly. Microwaves apply electromagnetic energy, generally to a significant depth, called “depth of penetration”, which depends on the properties of the material). The energy processes to the depth of penetration at nearly the same time. This is why there is a common belief that microwaves “heat from the inside out”, which is not the case for very thick sections of materials but it is true for thinner sections of materials (less than 2×the depth of penetration).

For a long time it was believed that microwaves accelerate a process by quickly adding heat throughout the material. Some examples of these thermal effects include; heating food, accelerating chemical reactions, removing moisture from materials, and staining tissues after histological tissues processing. But more recently it is becoming accepted that microwaves interact with some materials other than simply heating the materials, causing non-thermal effects. Some examples include desired grain growth in ceramic sintering, tissue processing and some chemical reactions. Generally the higher the microwave power, the faster the process, but the samples/material can become damaged if heated too quickly in a short period of time.

Since it is known that applying microwave to other processes accelerates them, the present invention applies microwaves to immunological testing would reduce the process (incubation) time. There exists literature suggesting the use of microwaves for accelerating the incubation times of immunological testing.

Procedures for Enhanced Microwave ELISA

Existing literature and experience indicates that using a standard off the shelf microwave to process ELISA assays result in lower resolution than the conventional 1 hour incubation times at room temperature as well as the reproducibility of the data was inconsistent. Also using these domestic microwave ovens each system needed to be “calibrated” or the microwave energy is buffered using water or other microwave adsorbing material to reduce the harmful overheating effect of microwaves. Therefore each oven needed to be calibrated or buffered individually.

Our first thought was that everyone was using too much power as most microwaves generate 800-1,100 watts and the antibodies or antigens where becoming damaged. This is true even when the microwave is on a lower power setting such as “medium”. Most microwave ovens adjust the power by cycling the power on and off, for example, a 1,000 watt microwave oven when on a power setting of “medium” (or 50% power) turns the microwave power on at 1,000 watts for about 4-8 seconds and then turns it off for about 4-8 seconds giving a time average of 50% power or 500 watts. This is called on-off duty cycling power adjustment. Often the 1,000 watts for 4-8 seconds damages the samples during the on time from too much heating. Think of dimming the room lighting by turning the light on and off for 6 seconds at a time

References

The following are offered as art in the field of the present invention, but non-disclose the present invention:

US Patents

| | | |
|---|---|---|
| 6498016 | Dec. 24, 2002 | Nahar, et al. |
| 5478748 | Dec. 26, 1995 | Akins, Jr., et al. |
| 5403747 | Apr. 04, 1995 | Akins, Jr., et al. |

US Patent application

| | | |
|---|---|---|
| 20050000811 A1 | Jan. 6, 2005 | Luka, Janos |

International Applications (PCT)

| | | |
|---|---|---|
| WO 2006/137945 A2 | Dec. 28, 2006 | Geddes, Chris |

Other References

Akins et al. “Measurement of Protein in 20 Seconds Using a Microwave BCA Assay”, Biotechniques, 12, 4 pp. 496-499.

Boon et al. Microwave Cookbook: The Art of Microscopic Visualization Coulomb Press Leyden, Leiden Netherlands 1989.

Gedye et al. The Use of Microwave Ovens for Rapid Organic Synthesis Tetrahedron Letters 1986 27:279-282.

Marani, E. “Microwave Applications in Neuromorphology and Neurochemistry: Safety Precautions and Techniques” A Companion to Methods in Enzymology, vol. 15, p. 87-99, (1998 Van Dorp, R., et al. “ELISA Incubation Times can be Reduced by 2.45-GHz Microwaves” J. Clin. Lab. Immunol., vol. 34, p. 87-96, (1991).

Van Dorp, R., et al. “A Rapid ELISA for Measurement of Anti-Glomerular Basement Membrane Antibodies using Microwaves” J. Clin. Lab. Immunol., vol. 40, p. 135-147, (1993).

Zhang et al. “Use of microwaves in Immunoenzyme techniques.” Clinical Chemistry, vol. 39, No. 9, 1993, p. 2021.*.

Zhang, L., et al. “Use of Microwaves in Immunoenzyme Techniques” Clinical Chemistry, vol. 39, No. 9, p. 2021, (1993).).

### SUMMARY OF THE INVENTION

Immunological testing as termed here means testing of blood, blood serum, bodily fluids or tissues for antibodies, antigen, enzyme reactions, In Situ Hybridization (also called FISH) or similar Immunological reactions common to Enzyme-linked Immunosorbent Assays (ELISA) (ELISA assays are also sometimes called Microtieter assays), protein blotting methods (commonly Western Blot, Immunohistochemistry or other Immunopercipitation methods. It does not include histostaining to improve imaging of cells performed on histological embedded tissues. Microwaves have been used for accelerating these types of processes for some time. Immunological testing includes the testing for HIV, certain cancers, allergies, diabetes and other diseases and is one of the most common laboratory testing performed in the medical field.

In the above Immunological testing, antigen or antibodies, proteins or enzymes are mixed and react (frequently bonding in antibody-antigen pairs) and must be given a period of time to react, called the “incubation time”. While there may be multiple incubation periods in each immunological assay, most of the incubation periods are 1 hour or longer. For example, there maybe 2 or 3 incubation periods within a given ELISA assay each at least 1 hour long. Typical incubation is done at room temperature or at slightly elevated temperatures in incubation ovens generally at 37° C. However the antibodies and/or antigens become denatured in temperatures above around 45° C.

Resolution is important in Immunological testing. Higher resolution means lower levels of undesirable antigens can be detected and equate to earlier detection of disease or viruses that can be treated with higher success rates than those detected later. Essentially, the assays combine certain antibodies and antigen and then the results are determined through the number of these combinations as detected in different methods depending on the technique used.

In a common ELISA assay called indirect, antigens are coated onto a “plate” with a number of small wells. The antigen is bonded to the walls in the wells. The fluid or serum to be tested is then placed into the wells and antibodies in the fluid or serum bonds to the antigen coated in the well during the incubation period. The plate is washed to remove the fluid or serum and unattached antibodies. Then the well is filled again with a fluid containing antibodies that have a detectable “marker”. These marker antibodies combine only with the antibody and antigen pair that is bonded together during the 1^{st} incubation period. After a second incubation period, the plates are washed again and then read to detect the number of markers (and paired antigen/antibodies) are left. See FIG. 1.

We have shown the present invention to reduce incubation times while retaining acceptable results in terms of resolution or sensitivity.

We have tested our enhanced microwave shortened incubation on:
Indirect ELSIA assays
Capture ELISA assays
Western Blot
Immunohistochemistry slides

All our testing has shown our enhanced microwave incubation has always shortened the incubation times with acceptable resolution equal to or better than traditional incubation methods and times (room temperature and 1+hour time). ELISA assays generally are evaluated by reading a color reaction using a special microplate reader, which measures the absorbance of the color intensity. However it can be also developed or read by other methods including chemiluminescence, radioactivity, or other means documented in the literature.

The following are additional benefits of the present invention:

Reduced incubation times—conventional ELISA assays or other immunological testing methods are typically incubated at room temperature for 1 hour or more for each incubation period. Indirect ELISA assays have 2 incubation periods and 2 washing, making the total time 2 hours +5 minutes for each washing or a total of about 2 hours and 10 minutes. Using the best protocol resulting from the “invention” each incubation time can be less than 5 minutes and the total ELISA assay process time using the “invention” to be 10 minutes total. Similar results have been shown with Western blotting and other immunological methods.

Resolution—Resolution is important as it leads to earlier detection of disease or illness and better potential for more effective treatment. The resolution using the “invention” is at least as good as conventional ELISA assays with 1 hour room temperature incubations. Similar results have been shown with Western blotting and other immunological methods.

Use standard components—There is no need for custom or special ELISA plates Standard ELISA plates can be used to reduce cost. This is also true that standard components can be used with the other immunological testing methods including Western blots.

No FDA approval required—Since the FDA does not specify incubation methods and standard test components (such as standard ELISA plates or Western Blot paper) are used, there is no requirement for approvals from the FDA, a long and expensive procedure.

Consistent incubation—With consistent incubation comes consistent processing and results, assay to assay as well as well to well within the assay plate.

### BRIEF DESCRIPTION OF THE DRAWINGS

FIG. 1 is a flow chart of the process of the present invention.

FIG. 2 is a flow chart of additional aspects of the present invention

FIG. 3 is a graph detailing a comparison or reproducibility of data using enhanced microwave and conventional indirect ELISA assays incubation.

FIG. 4 is a graph showing comparison of sensitivity of enchanced microwave ELISA versus conventional indirect ELISA assays incubation.

FIG. 5 is the result of conventional room temperature incubation Western Blot developed using the chemiluminescence method.

FIG. 6 is the result using the enhanced microwave Western Blot method developed using the chemiluminescence method.

### DETAILED DESCRIPTION OF THE PREFERRED EMBODIMENT(S) OF THE PRESENT INVENTION

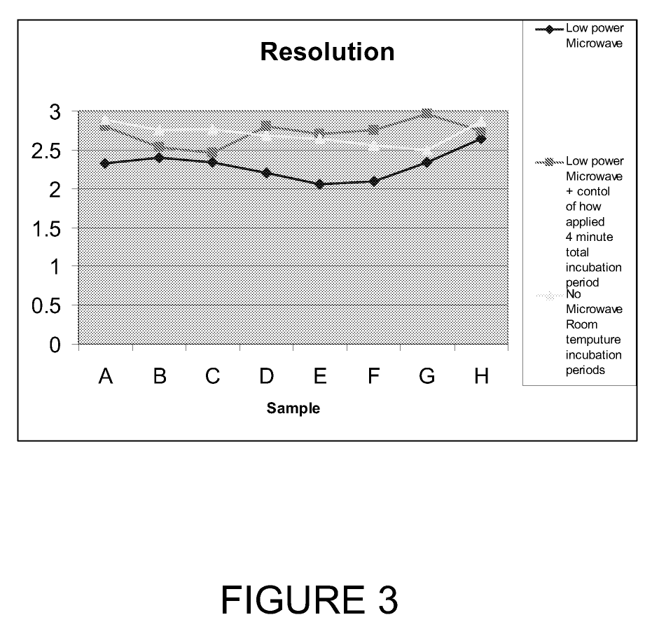

We used a microwave with a power control system that can change the actual power level without on-off duty cycling power adjustment, like a room dimmer switch. The reduced power was continuously applied and it was less than 300 watts for a single ELISA plate. The results showed that the resolution was still not as good as the room temperature ELISA assays. See FIG. 3, trace labeled “Low Power Microwave” (Blue diamond).

Although the antigen to antibody bonds are fairly strong, that once formed they need a short amount of time to stabilize into a stronger bond. We theorized that while the low power microwave aided in forming antigen to antibody bonds, they could still also break the bonds and separate the antigen and the antibodies, if the antigen to antibody pairs did not have a chance to strengthen their bonds.

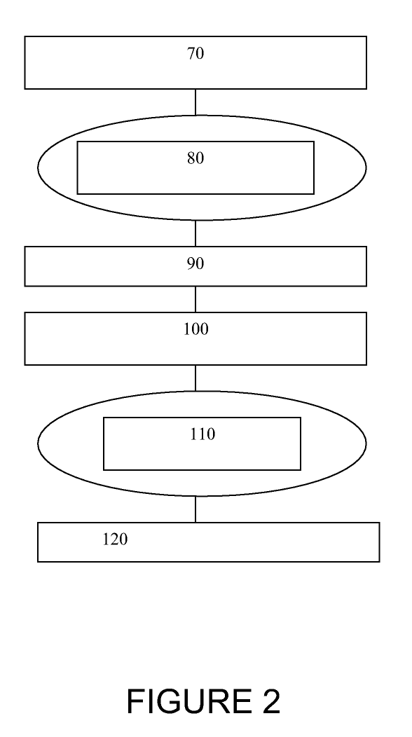

We then tested the idea of applying the low power level microwave energy specifically in such a way that it allows the paired antigen and antibodies the time to combine and then remove the microwave energy for a period of time to allow the formation of stable and strong bonds. We found that even repeating the cycle of applying power and a rest period worked well and yielded resolution equal to conventional room temperature incubation in less than 4 minutes with our enhanced microwave incubation, see FIG. 2. FIG. 3 (Purple line trace with squares) is data that show the well to well reproducibility of the enhanced microwave ELISA compared to conventional ELISA (FIG. 3 yellow trace with triangles)

The next step was to compare the conventional ELISA and Enhanced Microwave ELISA in a titration assay where the ELISA plate was coated with various concentration of the antigen antibody was diluted 1/2,000. This assay run in duplicate would demonstrate the sensitivity of the two assays.

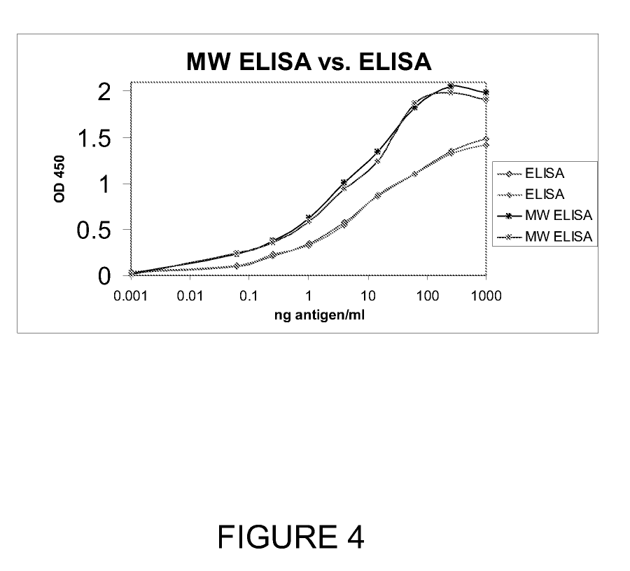

As shown in FIG. 4 the enhanced microwave ELISA (MW ELISA) was at least equally sensitive as the conventional room temperature ELISA (ELISA). In fact it was slightly more sensitive than the conventional ELISA.

Procedures for Microwave Enhanced Westerns

We further tested the enhanced microwave on Western Blots. Preliminary steps, prior to transfer, include, but are not necessarily limited to: gel electrophoresis of the sample(s); processing of the gel(s) prior to and in preparation for transfer (as applicable); preparation of the membrane(s) for transfer; preparation of filter paper and/or blotting paper and/or the like as needed; preparation of reagents, buffers and the like for one or more of the following, among others—transfer, washing(s), enhanced microwave reaction(s), background reduction step(s), and detection step(s), set up of device(s) for transfer and microwave enhancement, among others. Enhanced microwave Westerns employ the same gels, membranes, reagents and devices of conventional room temperature incubation Westerns.

Samples that can be analyzed by E3 enhanced microwave Westerns include the same range of samples that can be analyzed by conventional Westerns.

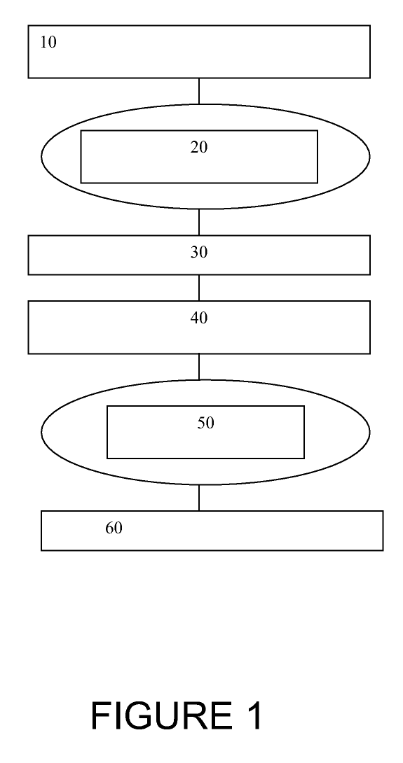

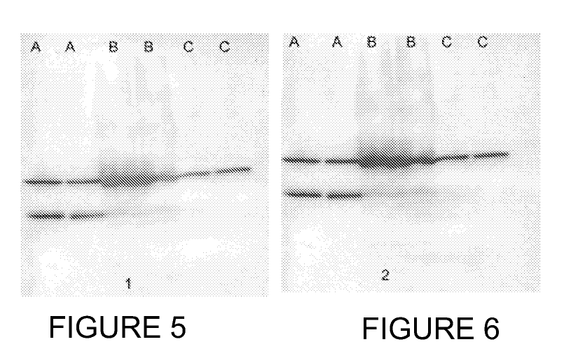

We transferred three different antigens (A, B, C) in duplicate to PVDF membrane after electrophoresis and using antibodies against the three antigens at 1/5,000 dilutions and performed the western blotting reactions with both the conventional room temperature incubation method (1 hour incubation with the first antibody and 1 hour with the secondary antibody) (See FIG. 1) and with the enhanced microwave method (See FIG. 2). FIG. 5 shows the result of conventional room temperature incubation Western Blot and FIG. 6 shows the result using the enhanced microwave Western Blot method. As shown by the pictures the sensitivity of the enhanced microwave method is as good as the conventional method.

It is understood and generally agreed that enhanced microwave Western methods provide significant advantages and improvements over conventional methods, including much shorter assay times, formation of stronger, more stable antibody-antigen pairing bonds, lower backgrounds readings, improved sensitivity, better and extended linearity, and increased reliability in many instances.

Gels are useful for carrying out enhanced microwave Westerns include those used to carry out conventional Westerns. The gels can be any gel for separating material to be probed by Western Blotting methods, such as agarose, polyacrylamide and mixed agarose polyacrylamide gels, in the main the same as for conventional Westerns.

Specific substances transferred from the gel are detected on the membrane in enhanced microwave Western methods using much the same reagents as conventional Westerns. In general, as for conventional Westerns, a primary antibody (or other target-specific binding reagent or probe) is used in a first binding reaction to bind specifically to a target bound to a membrane. Because the same reagents can be used, the enhanced microwave method is rapidly adaptable to replace the conventional Western method.

Specifications

The present invention is the method of applying a level of energy to reduce long incubation times, typically one hour or more at room temperature. The invention applies microwave energy for a period of time from 1 second to 2 minutes followed by a relaxation period from 1 second to 2 minutes where no energy is applied to allow the antibody/antigen or antibody/antibody pairing bonds to strengthen in immunological tests. There may be more than one period of applying microwave energy followed by more than one relaxation period followed by an extended relaxation period after the periods of applying energy and relaxation periods. Shortened incubation periods with resolution equal or better than conventional methods are obtained.

The number, power level and microwave energy applied and relaxation times change with different immunological tests. The best embodiment of the method is different for different types of immunological tests. The best embodiment of the invention applies microwave energy (2.450 MHz) to a standard 96 well ELISA plate (in this case coated with u94 peptide ( 1/1000) and monoclonal supernatant undiluted (3F3)) or a standard Western Blot holder.

Microwave: The MRA ImmunoBooster™ is a laboratory microwave, similar in size to your home microwave, which applies the proper power level during the energy applied periods for the proper time and the proper power level during the relaxation periods for the proper time. When using the ImmunoBooster™ for ELISA, there are holders to locate the ELISA plates, and likewise for Western Blot holders set the location in the microwave to be consistent.

The preferred embodiment of enhanced microwave for:

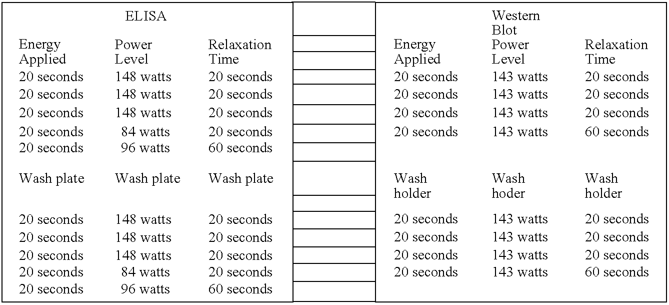

In our testing, the above enhanced microwave process (protocol) on the appropriate assays gives equal or better performance (resolution, consistency or clarity) as conventional room temperature incubation methods that take over 2 hours on the same type of immunological assay.

In regard to FIG. 1, the process is as follows: A user will add serum containing antibodies to antigen coated ELISA plate (10). Incubate˜1 hour at room temperature (20). Wash ELSIA plate (30). Add serum containing marking antibodies to ELISA plate (40). Incubate 1 hour at room temperature (50) and then read results with ELISA reader (60)

FIG. 2 adds the alternative embodiment of the present invention that a user will add serum containing antibodies to antigen coated ELISA plate (70) and Incubate 4 minutes in enhanced microwave (80). The user will then wash ELISA plate (90) and add serum containing marking antibodies to ELISA plate (100). Incubate 4 minutes in enhanced microwave (110) and then read results with ELISA reader (120).

In addition, we found that shortening incubation times on some immunoassays are more difficult than others. Monoclonal antibodies are easier to incubate than polyclonal antibodies, or they at least only need a single level of time or energy to bond. Polyclonal antibodies on the other hand are made up of many different antibodies that all may have different incubation requirements.

We have found that polyclonal antibodies do not bond as well (easily) as most monoclonal antibodies at the same energy level or incubation periods. We found that many polyclonal antibodies need longer time to incubate or we need to use higher energy (power) levels of enhanced microwaves. Unfortunately, with the higher energy (power) levels, the immunoassay becomes too heated and damaged.

We added cooling to the immunoassays to keep them sufficiently cool to prevent that damage. We cooled using liquid cooling and gas cooling.

Liquid Methods:

While liquid cooling methods offer better cooling (heat transfer) they may not be desirable because they can cause problems if spilled in the microwave. The liquid can absorb the energy intended for the immunoassay if the liquid absorbs microwave energy. They can also drip liquid into one Immunoassay when removing one Immunoassay, thus contaminating it.
1. Stop the enhanced microwave reduced incubation part way through the cycle and float the Immunoassay in a volume of liquid (water) then continue with the enhanced microwave reduced incubation. This has obvious drawbacks in the process requires interruption and operator intervention. It is also not consistent.
2. We then introduced a valve, drain system and spray system into the microwave cavity to spray liquid (water) onto the back of the Immunoassay during the rest periods of the enhanced microwave reduced incubation. The cooling can only occur during the periods where the microwave was off because if the liquid where being sprayed during the periods where the microwave is on, the energy intended for the immunoassay would be absorbed by the liquid intended for cooling and be counterproductive. We testing this using 2 or 3 rows of tubes made of non microwave absorbing materials (PTFE) with a series of holes to direct the spray of liquid to the bottom of the Immunoassays to cool them. The liquid was given sufficient time to drain out of the microwave before the microwave energy (power) was turned back on to continue pair bonding. In this test, water was supplied from the tap at typically 18-24° C. and drained into a drain
3. Other embodiments include the use of a recalculating pump and water chiller to reduce the temperature of the water to maintain 12-24° C.

Other embodiments includes have water flow under the Immunoassay in place of spraying it at the underside.

Gaseous Methods

Gaseous methods are preferred because they can be used even during the period of the application of the microwave energy (power) as most gasses do not absorb the microwave energy (power) and will not interfere with the microwave energy (power) intended for the immunoassay. They can be cooled to increase their cooling efficiency.
1. Using an air pump, we using 2 or 3 rows of tubes made of non-microwave absorbing materials (PTFE) with a series of holes to direct the spray of liquid to the bottom of the Immunoassays to cool them. This requires an air pump and the air must be forced through the tubes.
2. This method uses a fan or blower and ducting to direct the gas (air) to flow below the immunoassay cooling it during the entire enhanced microwave reduced incubation period. We intend to use two (2) 60 mm×60 mm Peltier (thermoelectric) coolers to dissipate up to 200 watts power turned to heat to pre-cool the gas (air) to further increase the cooling. The gas would simply be ducted to flow through the underside of the immunoassay (commonly a 96 well ELISA tieter plate. The gas (air) temperature could be lower than 0° C.

Best embodiment using air cooling chilled to +8° C.

ELISA

| Energy | Power | Relaxation |
|---|---|---|
| Applied | Level | Time |
| 10 seconds | 180 watts | 10 seconds |
| 10 seconds | 180 watts | 10 seconds |
| 10 seconds | 180 watts | 10 seconds |
| 10 seconds | 180 watts | 10 seconds |
| 10 seconds | 150 watts | 10 seconds |
| 10 seconds | 150 watts | 10 seconds |
| 10 seconds | 150 watts | 10 seconds |
| 10 seconds | 150 watts | 30 seconds |
| Wash plate | Wash plate | Wash plate |
| 10 seconds | 180 watts | 10 seconds |
| 10 seconds | 180 watts | 10 seconds |
| 10 seconds | 180 watts | 10 seconds |
| 10 seconds | 180 watts | 10 seconds |
| 10 seconds | 150 watts | 10 seconds |
| 10 seconds | 150 watts | 10 seconds |
| 10 seconds | 150 watts | 10 seconds |
| 10 seconds | 150 watts | 30 seconds |

We claim:

1. A method for accelerating the incubation periods of immunological testing, comprising:
applying a first burst of energy with a frequency in the 2.450 GHz ISM band to the immunological assays for a period of time from less than 1 second to 2 minutes;
following application of the first burst of energy by a first relaxation period from 1 second to 2 minutes where no energy is applied;
allowing antibody/antigen bonds to strengthen; and
allowing antibody/antibody pairing bonds to strengthen.
The method in claim 1, further comprising applying a second burst of energy after the first relaxation period.

2. The method in claim 2, further comprising following the second burst of energy with a second relaxation period.

3. The method of claim 3, further comprising allowing antibody/antigen bonds to strengthen.

4. The method of claim 3, further comprising allowing antibody/antibody pairing bonds to strengthen.

5. The method of claim 4, further comprising allowing antibody/antibody pairing bonds to strengthen.

6. The method of claim 2, wherein the second burst of energy has a frequency in the 2.450 GHz ISM band to the immunological assays.

7. The method of claim 2, wherein the second burst of energy has a duration of the period of time from less than 1 second to 2 minutes.

8. The method of claim 3, wherein the second relaxation period is from one second to two minutes.

9. The method of claim 1, wherein the first burst of energy is microwave energy.

10. The method of claim 2, wherein the second burst of energy is microwave energy.

11. The method in claim 1, further comprising applying microwave energy from 900 MHz through 300 GHz.

12. The method in claim 4, further comprising emitting the microwave energy power level 600 watts or less.

13. The method in claim 4, further comprising adding microwave adsorbing material to a microwave circuit to absorb a portion of the microwave energy in the microwave circuit.

14. The method in claim 4, further comprising lowering the energy down to 20 Hz when the energy is of lower frequency.

15. A method for accelerating the incubation periods of immunological assays by applying energy of a frequency of 20 Hz to 300 GHz to the immunological testing components for a period of time from less than 1 second to 2 minutes followed by a relaxation period from 1 second to 2 minutes where no energy is applied.

16. The method in claim 8 with more than one cycle of applying energy and relaxation periods.
